(19) United States
(12) Patent Application Publication
(10) Pub. No.: US 2022/0162675 A1
(43) Pub. Date: May 26, 2022

(54) METHODS AND KITS FOR THE ENRICHMENT AND DETECTION OF DNA AND RNA MODIFICATIONS AND FUNCTIONAL MOTIFS

(71) Applicant: Active Motif, Inc., Carlsbad, CA (US)

(72) Inventors: Benjamin F. DELATTE, San Diego, CA (US); Eddie W. ADAMS, San Diego, CA (US); Joseph M FERNANDEZ, Carlsbad, CA (US)

(73) Assignees: Active Motif, Inc., Carlsbad, CA (US); Active Motif, Inc., Carlsbad, CA (US)

(21) Appl. No.: 17/616,147

(22) PCT Filed: Dec. 23, 2020
(86) PCT No.: PCT/US20/66986

§ 371 Date: Dec. 2, 2021

Related U.S. Application Data
(60) Provisional application No. 62/953,080, filed on Dec. 23, 2019.

(51) Int. Cl. C12Q 1/6806; C12Q 1/6858

(52) U.S. Cl. CPC C12Q 1/6806; C12Q 1/6858

(57) ABSTRACT

Provided herein are methods for mapping modified nucleotide residues in nucleic acids. The methods include providing a nucleic acid sample in which non-target or target modified and unmodified nucleotide residues are converted to form of a different nucleotide (such a “C” being converted to “T”). Second strand synthesis is then performed on the converted nucleic acids using a set of anchored-base primers. Each primer in the set of anchored-base primers comprises one or more anchor bases at the 3′ terminus that are complementary to the target nucleotide (e.g., “G” or “CpG”), and a sequence of nucleotides selected from a set of sequences that could be a fully or partially degenerate set of sequences. For example, the sequence could be 5′-XnG-3′ and/or 5′-X(n−1)CG-3′, wherein X is any base, and n=2 to 25. Double-stranded nucleic acid products can be analyzed, for example by amplification and high throughput sequencing.

### REFERENCE TO RELATED APPLICATIONS

This application claims the benefit of the priority date of U.S. provisional application 62/953,080, filed Dec. 23, 2019 the contents of which are incorporated herein by reference in their entirety.

### BACKGROUND

Epigenetics refers to differences in phenotypes between cells and organisms that is not the result of genetic differences. Methylation patterns in DNA can result in epigenetic differences in phenotypes causing, for example, changes in gene expression patterns. Methylation in DNA typically occurs at cytosine residues. This includes, for example, methylation at the position 5 carbon. The forms of this methylation include 5-methylcytosine (“5mC”) and 5-hydroxymethylcytosine (“5hmC”). More oxidized forms of 5-methyl cytosines include 5-formyl cytosine (“5fC”) and 5-carboxycytosine (“5caC”). Methylation of cytosine typically occurs at CpG sites—where the nucleotide sequence is “CG”. CpG sites tend to occur in clusters, referred to as “CpG islands”. In humans, about 70% of genetic promoters include CpG islands. The presence of multiple methylated CpG sites in CpG islands of promoters causes stable silencing of genes. Methylation is known to be associated with cancer and aging. In cancer, gene silencing can be due to hypermethylation of promoter islands.

The mapping of methylation patterns in DNA has become an area of significant study. Several mappings are currently in use. A common approach of these methods is the conversion of various forms of cytosine into uracil in a DNA molecule, sequencing of the converted molecules, and comparison of the resulting sequences to sequences of unconverted molecules or to sequences in a genomic database by for example, mapping techniques.

One of the most popular methods of mapping methylation patterns is bisulfite sequencing. Treatment of DNA with bisulfite converts cytosine residues, but not 5-methylcytosine or 5-hydroxymethylcytosine residues, into uracil. Because this involves the conversion of the 4-amino group into a 4-carbonyl group, the process also is referred to as deamination. In second strand synthesis, G pairs with the introduced U and is propagated during amplification as “TA”, rather than “CG”. Upon mapping, the presence of “C” in a sequence represents an original unmodified 5-methylcytosine or 5-hydroxymethylcytosine. The presence of “T” represents an original “C” (or 5-formylcytosine or 5-carboxycytosine).

Variations on this strategy include the use of Ten-Eleven-Translocation methylcytosine dioxygenase (“TET”) and/or APOBEC3A (“A3A”). TET converts 5mC, 5hmC and 5fC into 5caC. Bisulfite can convert 5caC into uracil. A3A converts C and 5mC into uracil, but does not convert 5hmC, when paired with methods of protecting 5hmC groups, for example, by glucosylation. Glucosylation can be performed by, for example, T4 beta-glucosyl-transferase. Strategies can be devised for mapping 5mC or 5hmC, alone.

DNA treated by various deamination strategies can be sequenced to map methylation sites in DNA. One such method is whole genome sequencing. However, to the extent methylation patterns can be localized in the genome, whole genome sequencing can be inefficient. Methods for enriching DNA for DNA containing modifications, such as methylation, are known.

The existing epigenetics art includes a number of methods for enriching, sequencing and/or detecting certain nucleic acid modifications, e.g. methylation, such as:
1. Enrichment-based methods (MeDIP and MBD-Seq/MIRA-Seq/MethylCap-seq) that utilize modification-specific antibodies or proteins/protein-domains capable of specific recognition of methylated CpGs.
2. Whole genome bisulfite sequencing
3. Reduced representation bisulfite sequencing
4. Methylation-specific (q)PCR
5. Bisulfite-PCR

See, for example, “Methylation-specific PCR: a novel PCR assay for methylation status of CpG islands” PNAS (1996) by James G. Herman et al.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows an exemplary protocol for whole genome bisulfite sequencing (“WGBS”) and an exemplary protocol for anchored based sequencing.

FIG. 2 shows an exemplary protocol for anchored base bisulfite sequencing. This method enriches for nucleic acids having 5-methylcytosine and 5-hydroxy methylcytosine residues. Treatment of nucleic acid with bisulfite converts cytosine (“C”), formyl cytosine (“5fC”), and carboxy cytosine (“5caC”) into uracil. Methylcytosine (“5mC”) and hydroxy methylcytosine (“5hmC”) are not modified. Second strand synthesis is performed with a set of primers comprising a “G” residue at the 3′ end and a degenerate sequence of nucleotides. Resulting double-stranded nucleic acids are subject to amplification, library prep and sequencing.

FIG. 3 shows an exemplary protocol for anchored base TAB sequencing. This method enriches for nucleic acid molecules having 5hmC residues. Treatment of nucleic acids with a glucosylating enzyme protects 5hmC residues with a glucosyl group. Treatment of the protected nucleic acids with TET protein or catalytic domain converts 5mC and 5fC into 5caC residues. Bisulfite treatment converts cytosine and 5caC residues into uracil. Second strand synthesis is performed with a set of probes as per FIG. 2. Resulting double-stranded nucleic acids are subject to amplification, library prep and sequencing.

FIG. 4 shows an exemplary protocol for anchored base A3A sequencing. This method enriches for nucleic acid molecules having 5mC, 5hmC, 5fC and 5caC residues. Treatment of nucleic acids with TET protein or catalytic domain converts 5mC, 5hmC and 5fC residues into 5caC residues. A3A treatment converts cytosine residues into uracil. Second strand synthesis is performed with a set of probes as per FIG. 2. Resulting double-stranded nucleic acids are subject to amplification, library prep and sequencing.

FIGS. 5A and 5B show an exemplary protocol for click chemistry library prep. Nucleic acid molecules are subjected to bisulfite treatment (or other treatments as described herein). Anchored base probes, as described herein, linked to a tag, such as biotin, are used in second strand synthesis of the treated nucleic acid molecules. Such primers may also include an adapter sequence, for example comprising an Illumina P5 sequence. (FIG. 5A.) Double stranded molecules are denatured, and extended second strands, attached to the tag, are captured using a capture moiety (e.g., streptavidin). Captured molecules can modified to incorporate an adapter sequence on the 3′ terminus using click chemistry. This can involve introducing a 3′ terminal azide (N3) group and attaching an alkylated adapter through a 5′-3-triazole bond. The molecule are then subject to amplification using a set of primers complementary to the 5′ and 3′ ends of the molecule (e.g., comprising P5/P7 adapter sequences). The resulting molecules can be subject to analysis, e.g., nucleic acid sequencing. (FIG. 5B.)

FIGS. 6A-6E show an exemplary protocol for linear amplification anchored base bisulfite sequencing. Adapter molecules comprising hairpin loops, wherein the loop does not contain C, and including methylated C residues in the double strand stem (that will be refractory to deamination, denaturing, and non-specific anchor), and non−“C” residues in the loop, are attached to end repaired target nucleic acid molecules. Bisulfite, or other treatment of the nucleic acid molecules results in a loss of complementarity and denaturing. (FIG. 6A.) A set of probes as per FIG. 2a and a strand-specific isothermal polymerase, such as phi29 polymerase, having strong displacement activity is then used to perform rolling circle amplification on the circularized target molecule to produce a concatemerized molecule. Cytosine residues that have not been deaminated into uracil are incorporated in the extension product as “G”, while cytosine forms that have been converted to uracil residues are incorporated as “A”. (FIGS. 6B-C.) The amplified concatemer can be cleaved into individual molecules using a restriction enzyme that recognizes a sequence in the double strand stem of the hairpin loops. (FIG. 6D.) The individual molecules can now be subject to amplification, such as PCR amplification, to incorporate indices and other adapter elements. The resulting molecules can be subject to analysis, for example, DNA sequencing. (FIG. 6E). Note that deoxyGTP used in the rolling circle amplification can be labelled with a fluorophore, allowing one to measure modified cytosines by fluorometry.

FIG. 7 shows results from anchored base bisulfite sequencing on mammalian cells. This figure shows the enrichment of CpG sites, anchored on “Gs” throughout the genome. When G is at the sixth position in the primer, 75% of the time there is a C immediately upstream. This indicates CpG methylation, a result that is not compatible with chance.

FIG. 8 shows results from anchored base bisulfite sequencing on Drosophila SL2 cells. This figure shows two technical replicates of anchored base bisulfite sequencing on SL2 to cells, including a heat map and browser tracks. These results demonstrate the reproducibility of the technique as clear overlap in heat maps and genome browser tracks are observed.

FIG. 9 shows results from an experiment on E. coli K12 strain DNA comparing DNA immunoprecipitation sequencing (MeDIP-Seq) and Anchored-Base Bisulfite sequencing. In the DNA, the second “C” in the sequence CCWGG is methylated. A background motif, AASTT, is used as a control. As can be seen, the signal produced by the methylated base is significantly stronger in anchored base bisulfite sequencing than in MeDIP-Seq.

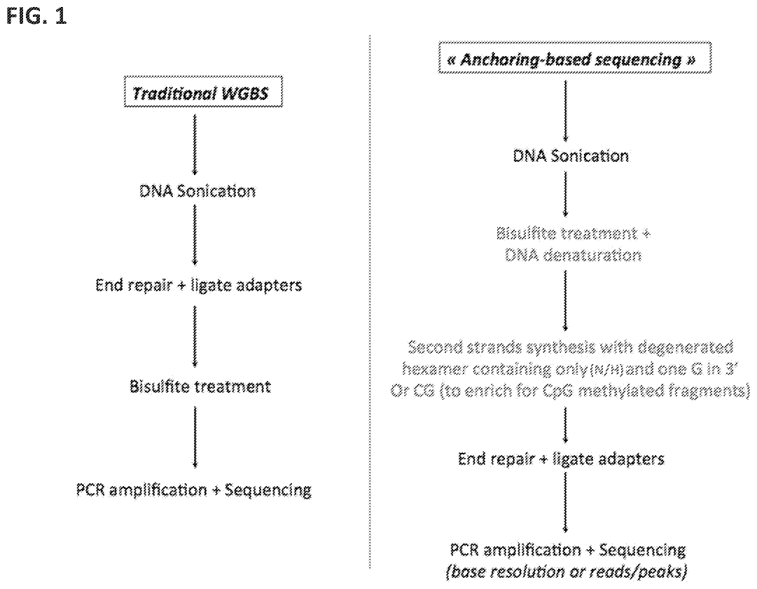

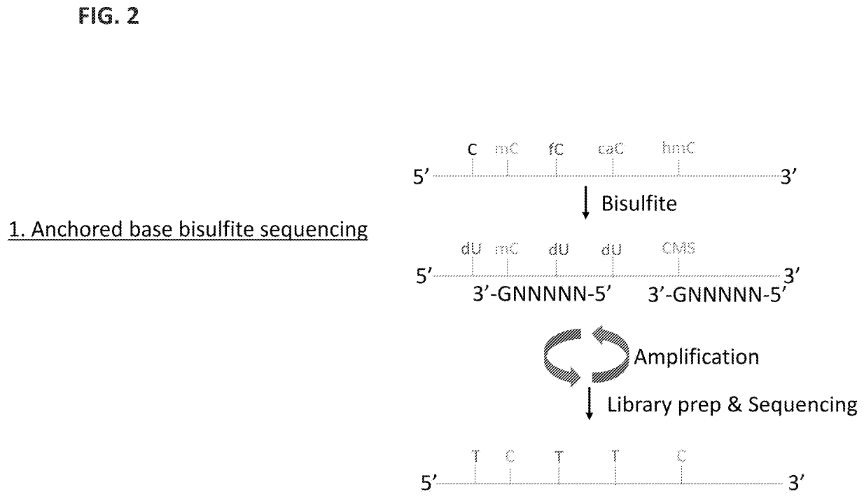

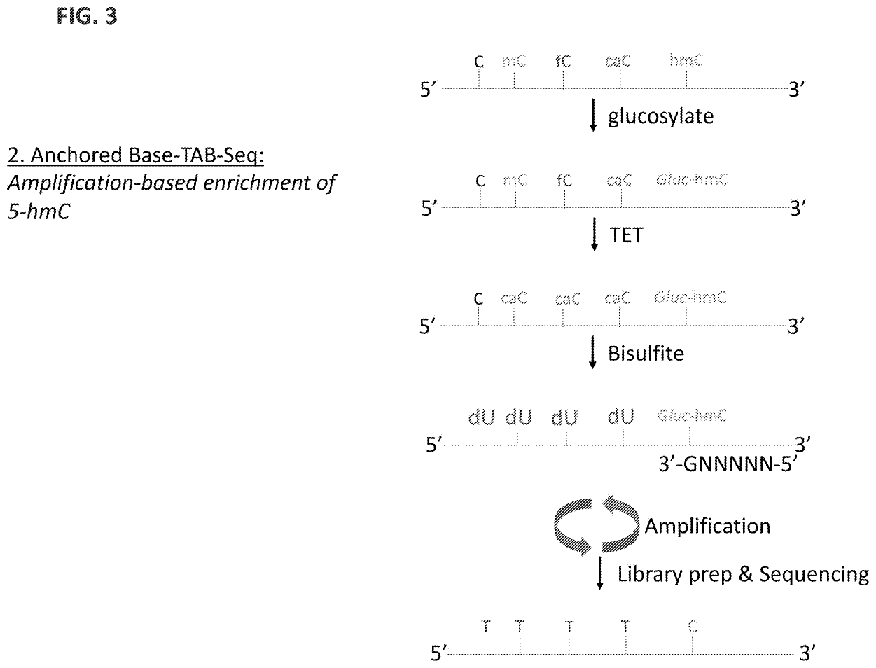

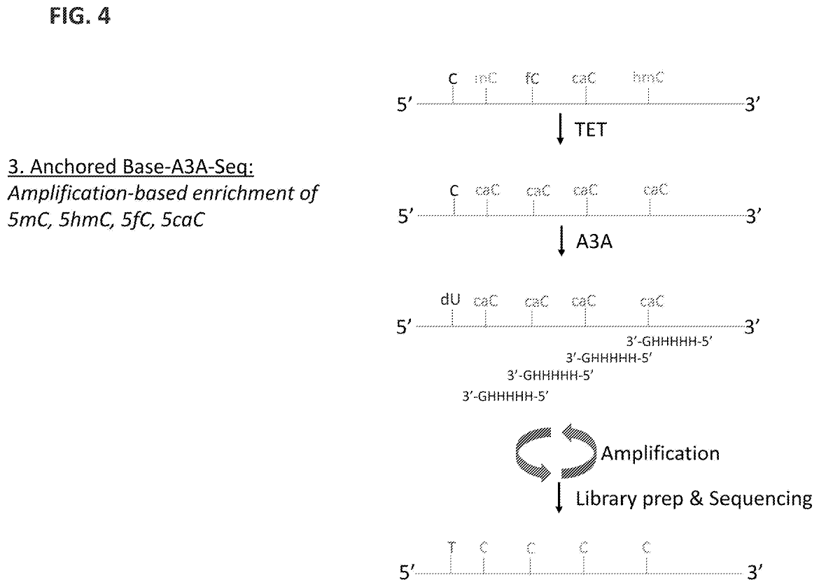

### SUMMARY

Provided herein are methods of analyzing nucleic acid molecules comprising modified bases. The methods involve converting a non-target base or bases in a nucleic acid, such as cytosine, into another base, such as uracil, and then performing second strand synthesis with a primer (typically a set of degenerate primers) having 3′ anchor base of G or CpG. The product of second strand synthesis is a set of double stranded nucleic acid molecules enriched for sequences containing the target base (such as methylcytosine or hydroxymethylcytosine) as a result of non-target bases having been converted to “U” which cannot serve as a template for a primer with the anchor “G”.

### DETAILED DESCRIPTION

### I. Introduction

Methods provided herein, in particular the ABBS embodiment, are superior to the existing art in a number of ways, including:
1.) They reduce the cost of DNA/RNA modification analysis via sequencing by enriching only those regions of DNA or RNA where the modification of interest is found—this means that one does not need to sequence as deeply to obtain high data coverage at the sites of interest.
2.) They simplify the design of diagnostic assays by coupling anchored-base random priming (a generic modification-biased amplification strategy) with basepair-specific probes.
3.) It simplifies the analyses, as one can use a peak-calling approach to map DNA or

RNA modifications, as well as bisulfite analyses (C→T transitions) on ABBS data since the method enriches for regions with potential high density of DNA/RNA modifications.

Disclosed herein are methods of enriching, identifying and mapping bisulfite-modified DNA throughout genomes of interest (e.g., bacterial, viral, human). The methods are also compatible with bisulfite-free methods of cytosine analysis as detailed below.

Four unique aspects to these methods, in particular the ABBS embodiment, compared to the existing art, include:
1.) Provision of a means of enriching and sequencing with single base resolution, modified cytosines (e.g., methylated CpGs) from the whole genome without having to use an antibody (as in MeDIP or hMeDIP) or modification-specific binding proteins (such as MBD2b/MBD3L in MIRA-Seq, and MeCP2 in MethylCap-seq);
2.) Provision of a means of amplifying and detecting modified cytosines without having to design specific primers against pre-determined sites within the genome (as in methylation-specific PCR assays)
3.) Ability to be incorporated into multiplexed diagnostic assays. By providing a highly generalized scheme of amplifying from sites of DNA/RNA modification, one only needs to design the specificity probe (e.g., a TaqMan hybridization probe) for the region of interest and not specific primer and probe sets for the region of interest. Thus, this strategy greatly simplifies assay design.
4.) Ability to be fully integrated with enzymatic methods of analyzing 5mC and 5hmC or other “oxi-mC” (5hmC, 5fC, and 5caC) distribution throughout the genome (e.g., TET-Assisted Bisulfite Sequencing and APOBEC-Coupled Epigenetic Sequencing (ACE-Seq)) whereas techniques like MeDIP, hMeDIP, MIRA-Seq or methylCap-seq cannot, since (1) they do not provide single-base resolution as in ABBS, (2) some protein may cross-react with other species (e.g. MeCP2 can also recognize 5hmC, not only 5mC), resulting in poor specificity, and (3) none of these techniques are universal as for ABBS that does not require protein or antibody.

Methods disclosed herein achieve the following:
1.) They enable a high throughput methodology of detecting, with base-pair resolution, bisulfite/APOBEC/AID-sensitive and insensitive DNA and RNA modifications.
2.) They provide a means of anchoring the amplification of nucleic acids at sites resistant to modification by bisulfite/APOBEC/AID-class of enzymes (e.g., methylated Cs for bisulfite, glucosyl-hmC and 5fC/5caC for AlD/APOBEC-class of enzymes) and facilitates rapid whole genome amplification from these bisulfite/APOBEC/AID-resistant loci.
3.) They enable efficient integration of common methods of molecular detection—e.g., TaqMan probes, Molecular Beacons, Padlock probes—that permit specific and multiplexed detection of DNA/RNA modifications.
4.) They enable the identification and tracking of bisulfite/Apobec3A-sensitive biomarkers in diagnostic and clinical applications.

Additional advantages, e.g. for the AB HiC embodiment, are discussed herein.

### II. Enriching Nucleic Acids for Sequences Comprising Modified Cytosine Residues

Methods provided herein allow for enrichment of nucleic acids having selected cytosine residue modifications. Enrichment allows for deeper sequence analysis and more efficient identification of modified residues. The methods can involve converting non-target forms of cytosine into non-cytosine nucleotide residues, and second strand synthesis of nucleic acid molecules comprising remaining cytosine-form residues using a set of degenerate primers having a “G” or “CG” residues at the 3′ location of the primer. The terminal nucleotide on the primer functions as an anchor from which extension proceeds. Because extension proceeds from unconverted cytosine residues, regions of the genome that include the target cytosine modification will be enriched.

A. Samples Comprising Nucleic Acids

Nucleic acids can be sourced from any biological sample, including, for example, from a virus, a cell or cells or microbiome of any living organism. This includes both prokaryotes (such as archaea and bacteria) and eukaryotes (such as plants, animals and fungi). Animals include, without limitation, insects, fish, amphibians, reptiles, birds and mammals. Mammals include, without limitation, carnivores (e.g., dogs and cats), artiodactyls (e.g., cattle, goats, sheep, pigs), lagomorphs (e.g. rabbits), perissodactyls (e.g., horses), rodents (e.g., mice, rats), and primates (e.g., humans and nonhuman primates (e.g., monkeys, chimpanzees, baboons, gorillas).

Nucleic acids can come from a cell line, a tissue, an organ or a bodily fluid. Cells from any organ or organ system of an animal. Such organs include, without limitation, heart, brain, kidney, liver, lungs, muscle, blood. Body fluids that can be sources of nucleic acids include, without limitation blood, plasma, serum, saliva, sputum, mucus, lymphatic fluid, urine, semen, cerebrospinal fluid or amniotic fluid. Organ systems include, without limitation, muscular system, digestive system, respiratory system, urinary system, reproductive system, endocrine system, circulatory system, nervous system, and integumentary system. A sample can be prepared, for example, by biopsy. This includes both solid tissue biopsy and liquid biopsy. The sample can comprise cell-free DNA (“cfDNA”), such as circulating tumor DNA. Nucleic acid fragments can have a length between about 100 to about 800 nucleotides or 350 to 450 nucleotides, e.g., around 400 nucleotides. cfDNA typically has a size of about 120-220 nucleotides.

Samples comprising nucleic acids can be sourced from a subject having or suspected of having a pathological state. Such states include, without limitation, hyperplasia, hypertrophy, atrophy, and metaplasia, including, e.g., cancer (e.g., a cancer biopsy sample). Other pathologies include neuronal diseases (e.g., Alzheimer's Disease, Amyotrophic Lateral Sclerosis, Creutzfeldt-Jakob Disease, Friedreich's Ataxia, Multiple Sclerosis).

Nucleic acids can be naked nucleic acids, that is, with no proteins attached. Alternatively, nucleic acids can be in the form of chromatin. As used herein, the term “chromatin” refers to a complex of DNA and histone and/or non-histone proteins.

Samples comprising nucleic acids can be sourced from a subject having a particular chronological age. Methylation patters are associated with age and, therefore, can predict premature or retarded aging.

DNA can be purified in the form of chromatin. DNA from chromatin can be enriched by methods such as chromatin immunoprecipitation (ChIP) and transposon-assisted chromatin immunoprecipitation. ChIP methods typically involve crosslinking chromatin in order to covalently bind proteins to nucleic acids. Chromatin can be crosslinked while still in the cell. The chromatin then can be sheared. Nucleic acids having particular proteins bound thereto, such as histones, can be immunoprecipitated using an antibody directed against the target protein. In transposon-assisted chromatin immunoprecipitation, the antibody against the target protein is bound, directly or indirectly, to a transposome. A transposome comprises a transposase attached to a transposon. Upon finding its target, the transposon is inserted into the DNA. When transposons are provided with primer binding sites, nucleic acid positioned between the primer binding sites can be amplified. (See, for example, U.S. Pat. No. 10,689,643, Jelinek et al.)

B. Nucleotides and Modified Forms Thereof

Nucleotides in RNA and DNA can exist in their native form or in various modified forms. Cytosine can exist in several different forms.

The term “modified nucleotide” refers to a derivative of cytosine, adenine, guanine, thymine or uracil. The term “modified cytosine” refers to a derivative of cytosine, typically derivatized with a chemical moiety at position 5. Exemplary modified cytosines include, in increasing order of oxidation state, 5 methylcytosine (“5mC”), 5 hydroxymethylcytosine (“5hmC”), 5 formylcytosine (“5fC”) and 5 carboxylcytosine (“5caC”). Another modified form of cytosine is N-4-acetyldeoxycytidine (“N4-acdC”). (See, e.g., international patent application PCT/US 2020/066741, filed Dec. 22, 2020.)

Reference to a nucleotide, in contrast to a base, by letter, can refer to either the “ribo” version or the “deoxyribo” version, unless otherwise specified. In general, nucleotides in DNA will be in the “deoxyribo” version, while nucleotides in RNA will be in the “ribo” form.

In certain methods disclosed herein the 4-amino group on cytosine can be converted to a carbonyl group. This process is referred to as “deamination”. In this instance, the base is now uracil. Deamination of cytosine or a modified cytosine by the replacement of the amino group with a carbonyl group at position 4 converts cytosine or a modified cytosine into uracil.

C. Conversion Strategies

Methods of detecting a particular base modification, such as methylation or hydroxymethylation, in nucleic acids can involve converting non-target forms of the base and/or modified forms of the base, into a base or base form other than the original base. As used herein, a “non-target” form of a base refers to a subset of the possible forms of a base. For example, in the case of cytosine forms, “5hmC” may be a “target” form, and “C”, “5mC”, “5fC” and “5caC” may be non-target forms. In other embodiments, “5mC” and 5hmC″ may be a “target” forms, and “C”, “5fC” and “5caC” may be non-target forms. A “non-base” residue, for example, a “non-cytosine” residue, refers to a different base form. For example, a “non-cytosine” base typically will be uracil, but could include guanine, adenine, or thymidine, and modified forms thereof. Several conversion strategies are known.

1. Bisulfite Sequencing

Bisulfite treatment of nucleic acids converts cytosine form residues other than 5mC and 5hmC into uracil by a process of deamination. Upon sequencing, 5mC and 5hmC (“target forms”) read out as cytosine, while unmethylated cytosine, formyl and carboxyl-cytosines (“non-target form”) read out as thymine.

2. TET Sequencing

Ten-Eleven-Translocation methylcytosine dioxygenase (“TET”) converts 5mC, 5hmC and 5fC into 5caC. It is available from a number of different species, including human, mouse, or invertebrate (e.g., Naegleria, Drosophila (dTet, also named DMAD or CG43444)). Mammalian TET includes TET1, TET2 and TET3. The TET enzymes each harbor a core catalytic domain with a double-stranded β-helix fold that contains the crucial metal-binding residues found in the family of Fe(II)/α-KG-dependent oxygenases. These catalytic domains also can be used in conversion steps. Accordingly, “TET” refers to the whole enzyme or a functioning catalytic domain, unless otherwise specified.

This enzyme can be used in a method for detecting the 5hmC residues in nucleic acid. The method can proceed as follows. 5hmC residues in the nucleic acid are protected by glucosylation. This can be done, for example using recombinant phage T4 beta-glucosyltransferase. Next, the nucleic acid is treated with a TET enzyme (usually TET1 or NgTET homolog from the protist Naegleria gruberi), which converts unprotected forms of cytosine, including cytosine, 5mC, and 5fC, into 5caC. Further treatment of the nucleic acid with bisulfite converts 5caC into uracil. Upon sequencing, 5hmC (“target form”) reads out as cytosine while other cytosine forms (“non-target form”) read out as thymidine.

3. A3A Sequencing

The AID/APOBECs are a group of cytidine deaminases that can insert mutations in DNA and RNA by deaminating cytidine to uridine. Enzymes from the AID/APOBEC family include the following human enzymes: APOBEC1, APOBEC2, APOBEC3A (“A3A”), APOBEC3B, APOBEC3C, APOBEC3D, APOBEC3F, APOBEC3G, APOBEC3H, APOBEC4, Activation-induced (cytidine) deaminase (AID). These enzymes convert cytosines and 5mC into uracil but do not modify (or with extremely low efficiency) 5hmC, 5fC or 5caC. This class of enzymes can be used in methods to detect modified forms of cytosine, without differentiating among them. In one version of the method, nucleic acids are first treated with TET enzyme which oxidizes 5mC, 5hmC and 5fC to 5caC. Subsequent treatment with A3A converts cytosine to uracil while 5caC remains resistant to conversion. Upon sequencing, 5mC, 5hmC, 5fC and 5caC (“target forms”) read out as cytosines, while natural non-modified cytosine (“non-target form”) reads out as thymidine.

D. Second Strand Synthesis

After non-target nucleotides in a nucleic acid molecule have been converted to non-base (e.g., non-cytosine) residues, nucleic acids comprising target nucleotides can be enriched by second strand synthesis anchored at the unconverted sites. Second strand synthesis comprises hybridization of a primer or primer set to the converted nucleic acid molecules, followed by primer extension using a polymerase. In certain embodiments, the polymerase has 5′-3′exonuclease and/or a strand displacement activity. Because the primers hybridize at target sites in the nucleic acid, the double-stranded molecules will be enriched for those containing target nucleotides.

1. Anchored Extension Primers

Extension primers used in the methods described herein can comprise a nucleotide sequence of: 5′-Xn-G-3′, or 5′-X(n−1)-CG-3′, wherein “X” is any base. “G” is positioned at the 3′ terminus of the molecule. In some embodiments, “n” is between 2 and 25, 12 and 25, 3 and 10, 4 and 7, or about 5 (e.g., the priming sequence is a hexamer). Primers can be provided individually. However, typically, they are provided as a set to be used together in a single second strand synthesis operation.

“X”, at any position, can be any of: “N”=A,C,T/U,G; “H”=A,C,T/U; and “I”=Irregular bases such as (1) regular bases (A,C,T/U,G) that are modified on the base (“Q”), or (2) universal bases (“J”). As used herein, a “universal base” is a base that binds with more than one standard base and, therefore, functions as a degenerate base. Exemplary universal bases are (deoxy)inosine, nebularine, 3-Nitropyrrole, 5-Nitroindole.

So, for example, in one embodiment, the primers in the primer set are hexamers having the sequence 5′-XXXXXG-3′ or 5′-XXXXCG-3′; 5′-NNNNNG-3′ or 3′-NNNNCG-3′; 5′-IIIIIG-3′ or 5′-IIIICG-3′; 5′-QQQQQG-3′ or 5′-QQQQCG-3′; 5′-JJJJJG-3′ or 5′-JJJJCG-3′ or any combination of these bases.

A set of primers including “Xn” or “X(n−1)” can comprise a degenerate set of sequences. A degenerate primer set is a collection of oligonucleotide molecules having sequences in which some positions contain a number of defined possible bases, resulting in a population of primers with similar sequences that cover all possible selected nucleotide combinations at the variable positions. For example, a degenerate set of primers having a sequence 5′-NNNNNG-3′ will include a primer in which each of the four canonical nucleotides (A, C, G, T/U) can be present at each position occupied by “N”. Such a set of sequences would be fully degenerate.

Alternatively, the primer set can be partially degenerate, or biased. For example, certain bases in the set can be overrepresented compared to random. For example, the base “C” may be present more frequently than random. This would be the case if one wants to use a transcription factor motif as part of the primer, in order to analyze cytosine modifications on this motif in a genome-wide manner.

Several primer design programs are available (e.g., OLIGO, OSP, Primer Master, PRIDE, Primer3, among others). These programs can design primer sets Taylor to specified criteria, such as C/G content.

In other embodiments, the sequence “Xn” or “Xn−1” represents a target nucleic acid motif sequence of interest. For example, the motif sequence can be “GAGG”, which is reverse-complementary to CCTC, a motif for transcription factors. The motif could be for a transcription factor such as NF-κB, CTCF, BORIS, YY1, TBP, AP-1, CEBP, HOX proteins.

Primers can be provided with auxiliary sequences including, for example, one or more of adapter sequences, sample barcodes and molecular barcodes. So for example, the primer could have the sequence 5′-[adaptor sequence]-[sample barcode]-[molecular barcode]-Xn-G-3′, or 5′-[adaptor sequence]-[sample barcode]-[molecular barcode]-X(n−1)-CG-3′.

In certain embodiments, primers can comprise sequencer-platform specific adapter sequences. Such sequences typically will include amplification primer sequences. For example, in Illumina sequencer adapters include the p5 and p7 sequences.

Sample barcodes are nucleotide sequences used to distinguish nucleic acid molecules originating from different samples, but typically sequenced in a single sequencing operation. Different samples are tagged with different barcode sequences. Typically sample barcodes are between about 6 and about 20 nucleotides.

Molecular barcodes are a set of barcodes used to differentiate original molecules in a sample. Nucleic acid molecules in a sample can be uniquely barcoded, which is to say, each molecule has a different barcode attached. Alternatively, the nucleic acid molecules can be non-uniquely barcoded, which is to say, the number of different barcode sequences used to tag molecules in the sample is fewer than the number of unique molecules in the sample. In the case of unique barcodes, sequence reads of molecules amplified from the same original molecule will share the same barcode, and can be distinguished thereby. In the case of non-unique barcodes, sequence information from the barcode and from target molecule can be used to determine sequence reads amplified from the same original molecule. Molecular barcodes are typically between about 6 and about 20 nucleotides.

Extension primers used in the methods disclosed herein can comprise any form of nucleic acid or nucleic acid analog compatible with function as a primer. This includes primers comprising, without limitation, DNA, RNA, locked nucleic acids (“LNA”), peptide nucleic acids (“PNA”), polynucleotides comprising modified bases, riboses, deoxyriboses, modified sugars, and polynucleotides comprising noncanonical nucleotides, e.g., other than A, T, C, G or U. Examples include, without limitation, universal base analogues such as inosine or nitroindole.

In other embodiments, primers can comprise sequences for function as a molecular inversion probe or a padlock probe. For example, the primer can comprise the priming sequence, 5′-Xn-G-3′, or 5′-X(n−1)-CG-3′, a second nucleotide sequence that hybridizes to a target nucleotide sequence positioned at the 5′ terminus of the molecule, and a linker sequence positioned between the priming sequence and the second sequence.

2. Primer Extension

From the converted nucleic acids, the practitioner creates a population of double-stranded nucleic acids enriched for sequences comprising target modified nucleotides. This process involves denaturing the converted nucleic acids to provide single-stranded nucleic acids. A primer set comprising an anchor base “G” or bases “CpG” at the 3′ terminus is contacted with the denatured nucleic acids under hybridization conditions and allowed hybridize.

The primers are extended using an appropriate polymerase. The polymerase can be a mesophilic or thermophilic polymerase. For example, the polymerase can be Klenow exo-polymerase, Klenow polymerase, DNA polymerase I, T4 DNA polymerase, Phi29 DNA polymerase, BST DNA polymerase, Taq polymerase, pfu polymerase and reverse transcriptases (e.g., Moloney Murine Leukemia Virus (M-MLV), Avian Myeloblastosis Virus (AMV), and their mutated/altered versions. In certain embodiments the polymerase has 5′-3′exonuclease or strand displacement activity. In this way, if several primers hybridize in proximity to one another, the primer that hybridizes furthest upstream of the others will create the longest extension product by digesting or displacing elongating polynucleotides hybridized downstream of the primer.

In the case of reverse transcription of RNA, one can employ dUTP nucleotides. The dUTP containing strand will not be amplified during library preparation, thus preserving the strand information for RNA-seq.

The product of primer extension will be a collection of double-stranded polynucleotides enriched for sequences comprising a modified base. This collection can be subject to library preparation.

E. Library Preparation

1. Isolation of Double-Stranded Nucleic Acids

Double-stranded nucleic acids may be separated from remaining single-stranded nucleic acids in a number of ways. In one embodiment, the composition can be subject to a single-strand nuclease, such as, but not limited to, nuclease S1 to digest single-stranded molecules. In another embodiment, single-stranded nucleic acids and double-stranded nucleic acids can be fractionated from one another using known methods. In one such embodiment, DNA is isolated using silica or non-silica-based methods that have high affinity for double-stranded nucleic acids and low affinity for single-stranded nucleic acids, such as silica particles and hydroxyapatite. These can involve binding DNA to silica particles or membranes, or DNA grade Bio-Gel HTP hydroxyapatite, and separating from other contaminants. In one embodiment, double-stranded nucleic acids can be specifically enriched by the use of double-stranded nucleic acid binding proteins such as anti-double-stranded DNA anti-idiotypic antibodies. In one embodiment, single-stranded nucleic acids can be removed (negative selection) by single-stranded nucleic acid binding proteins such as anti-single-stranded DNA anti-idiotypic antibodies. In one embodiment, primers are provided with a capture moiety such as, for example, biotin or desthiobiotin. Accordingly, double-stranded molecules created through primer extension will be biotinylated. These molecules can be isolated through capture with a partner for the capture moiety, such as streptavidin, and single-stranded DNA molecules can be digested by single-strand nuclease, such as, but not limited to, nuclease S1.

After end repair and adapter ligation, target nucleic acid sequences can be isolated using capture sequences. Capture sequences are polynucleotides comprising a nucleotide sequence capable of hybridizing to nucleic acid molecules having a target sequence. Once hybridized, the target sequences capture the hybridized sequences. Typically, probes will comprise a capture moiety, such biotin, or will be attached to a solid support, such as a magnetically attractable particle, to allow for separation of the bound material from unbound material.

2. End Repair and Adapter Ligation

Polynucleotides subjected to fragmentation, or cell free DNA typically comprise ends with single-stranded overhangs that require end repair before adapter ligation. End repair can be accomplished by, for example, an enzyme such as Klenow polymerase which cleaves back 5′ overhangs and fills in 3′ overhangs. The result is a blunt ended molecules. Adapters can be attached to blunt end DNA directly by blunt end ligation. Alternatively, the blunt ended molecules can be “A tailed” in the 3′ ends to produce a single nucleotide “A” overhang. Sequencing adapters having a single “T” overhang in the 5′ ends can therefore be attached.

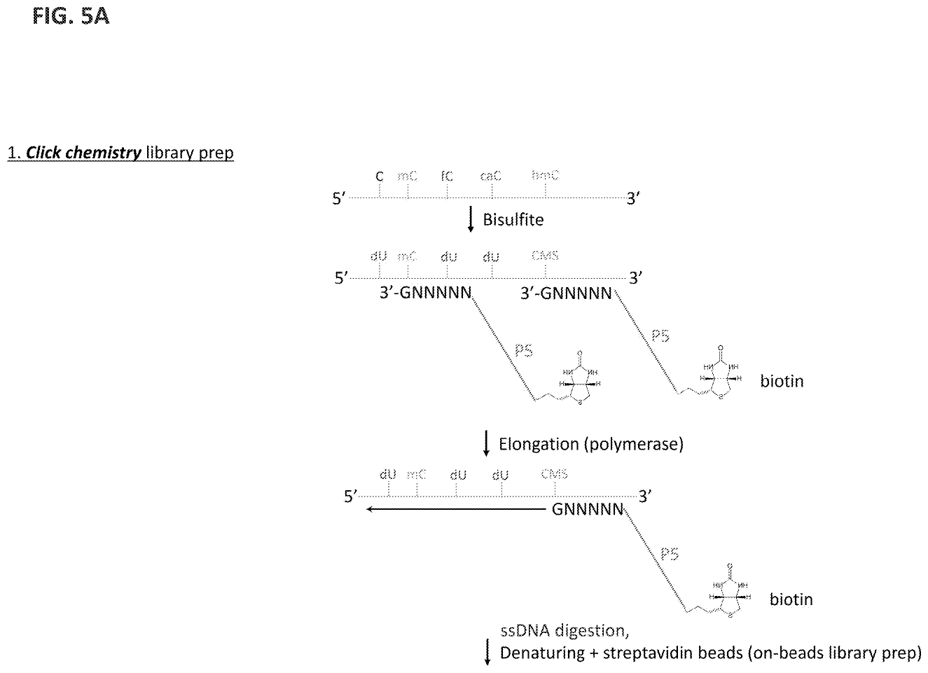

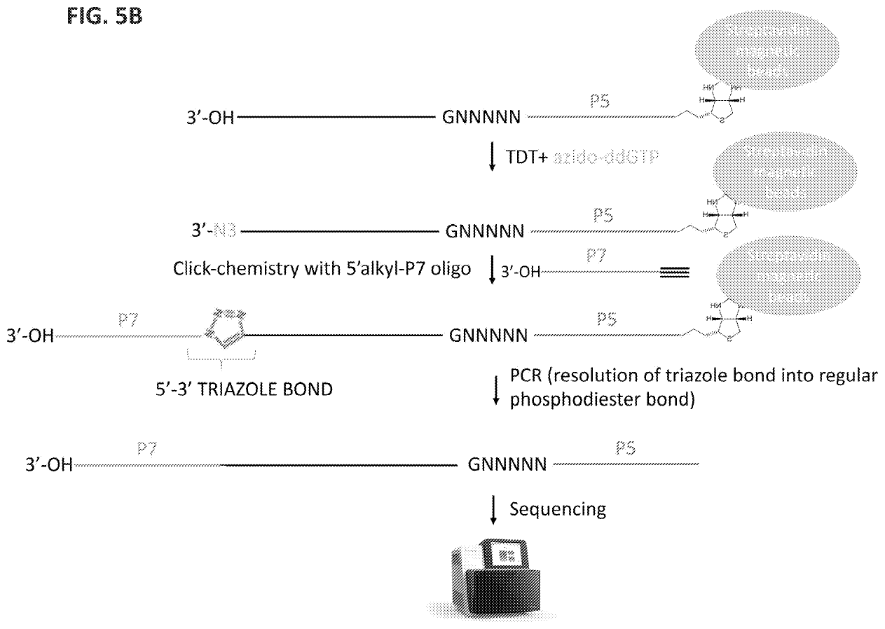

Alternatively, as discussed above, target polynucleotides can be provided with adapters through a primer extension reaction in which a primer molecule, as described herein further comprises adapter sequences In this instance, after elongation by a polymerase, DNA is tagged at the 3′ end with an azido ddNTP. Then an adapter containing an alkyl 5′ can be attached by click chemistry. DNA can then be PCR-amplified and further analyzed. (See, e.g., FIGS. 5A-B).

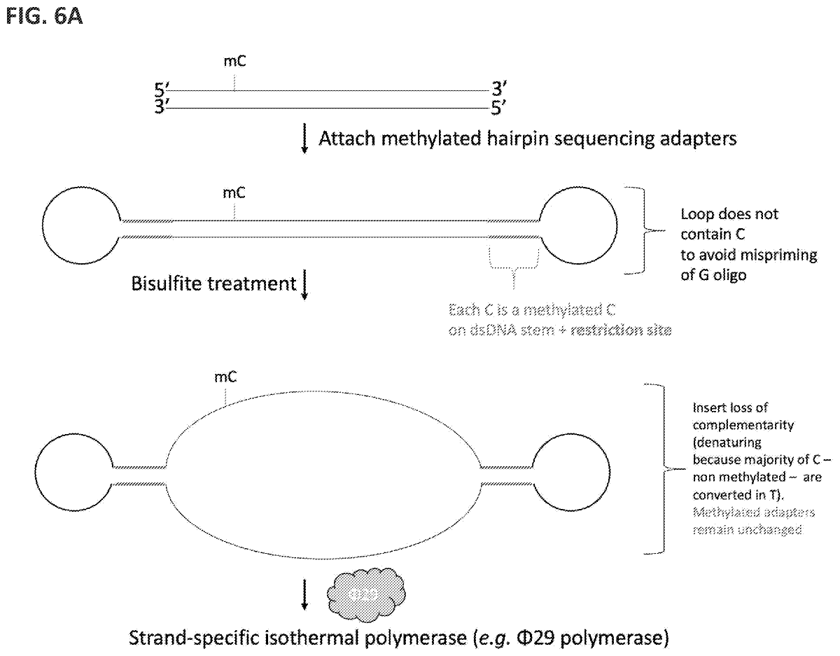

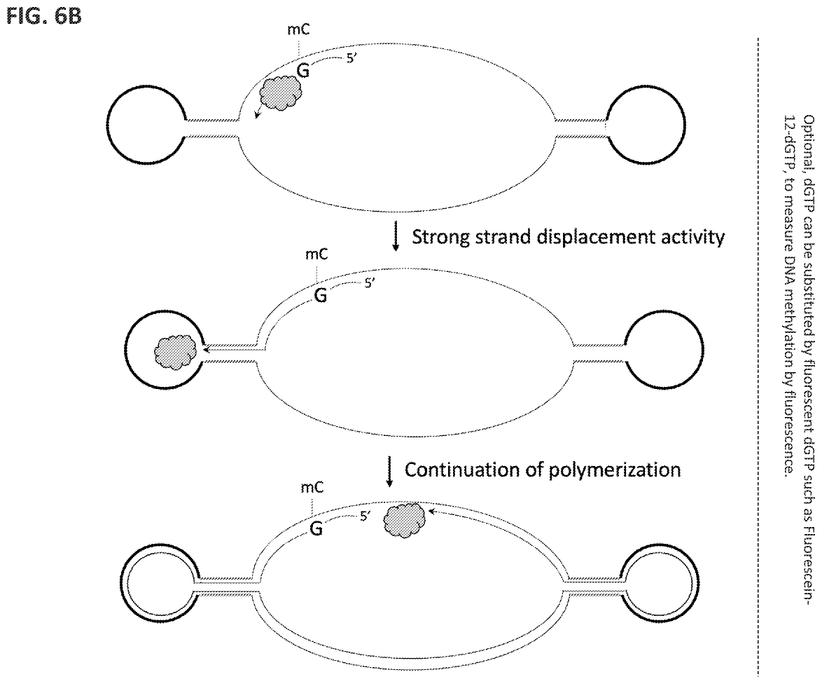

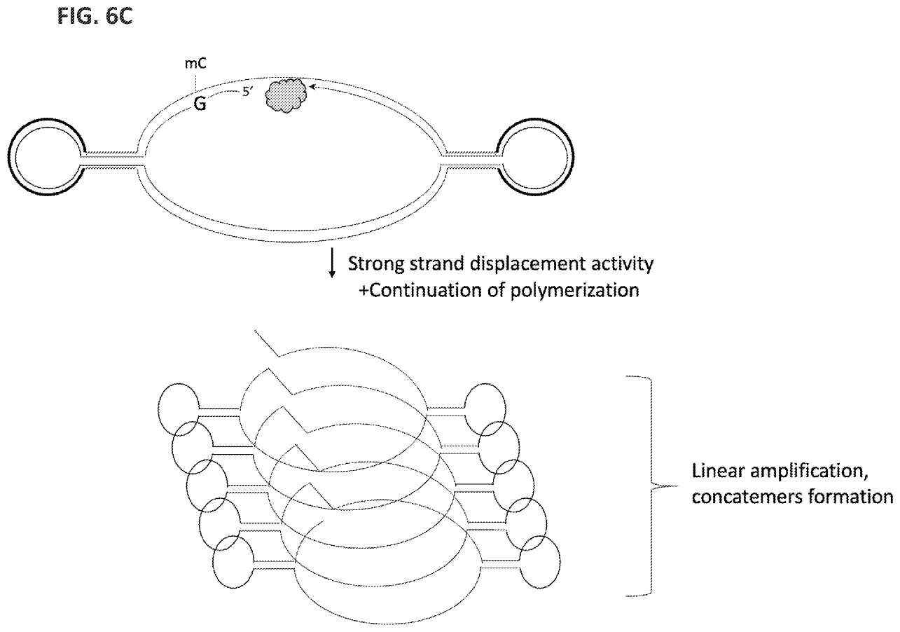

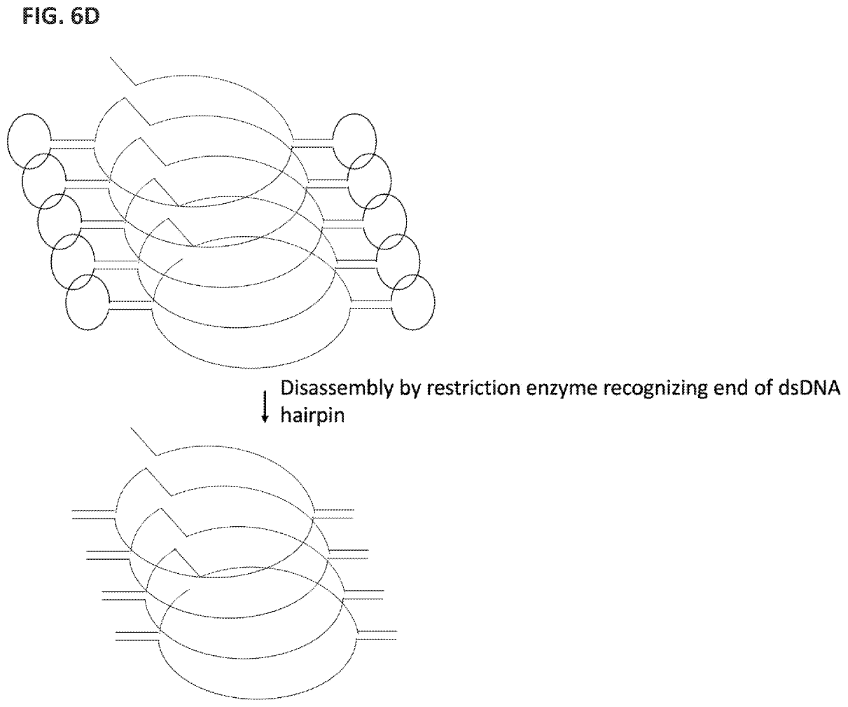

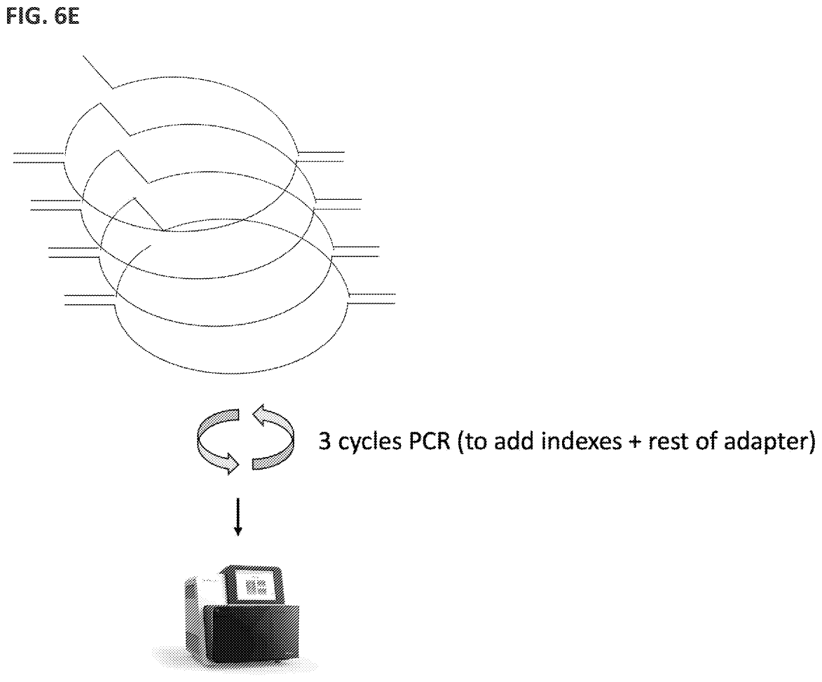

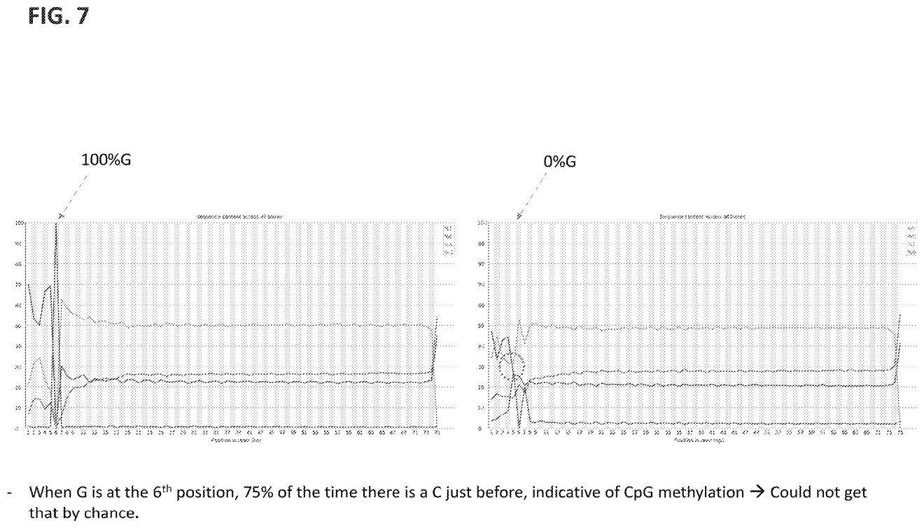

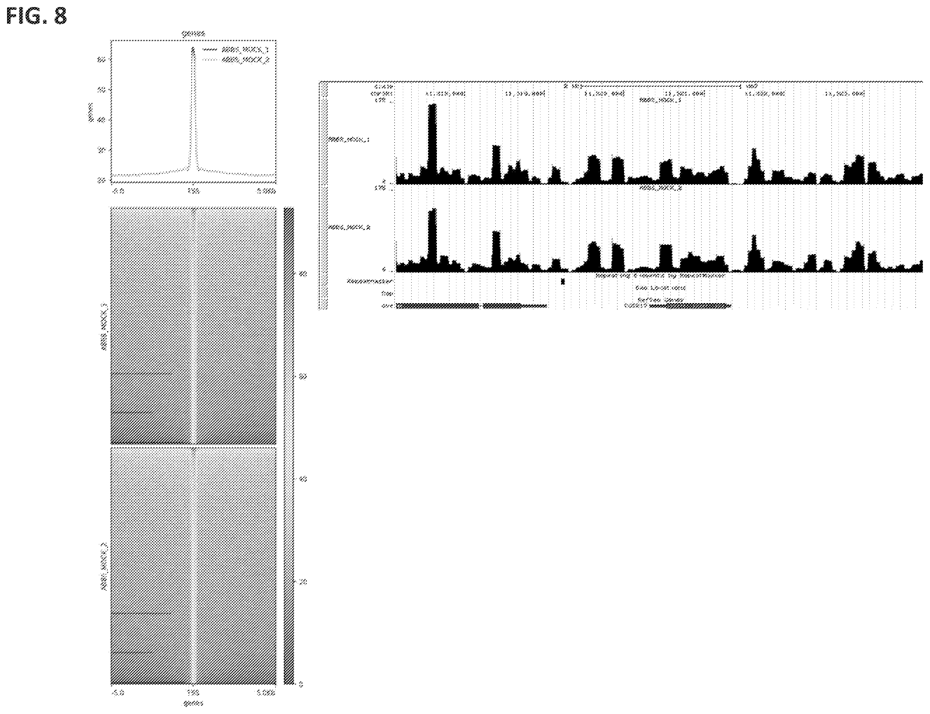

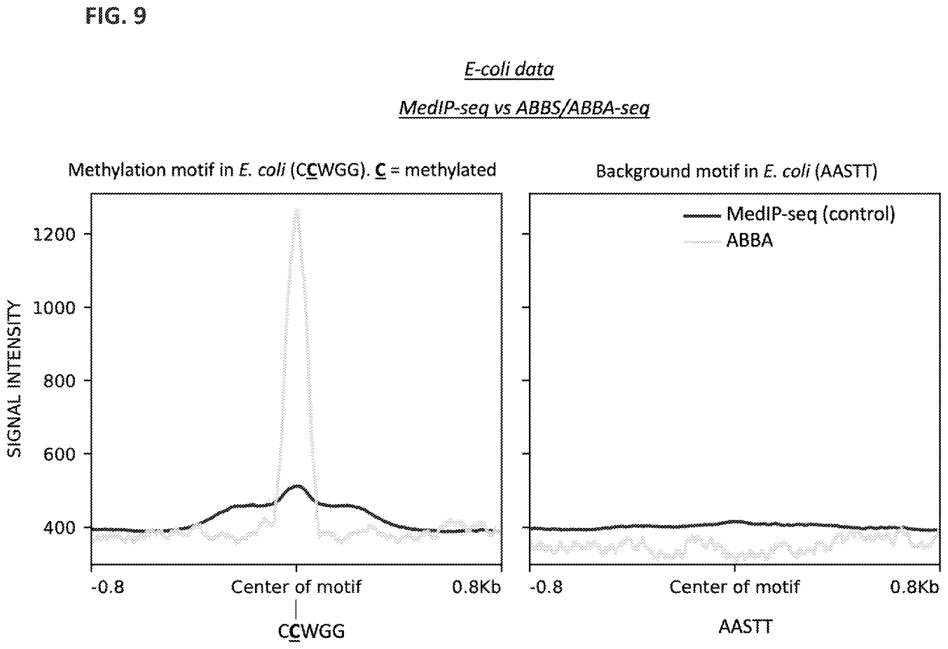

In another embodiment, adapter molecules comprising hairpin loops, including methylated C residues in the double strand stem are ligated, then after bisulfite and primer anchoring, a “rolling circle”-mediated library is created using an enzyme that contains a strong displacement activity such as Phi29/ϕ29 polymerase (See, e.g., FIGS. 6A-E).

It is noted that auxiliary sequences, such as sequencer primer sequences, sample barcodes and molecular barcodes can be provide in adapters ligated to double stranded molecules.

3. Nucleic Acid Amplification

Double-stranded nucleic acids can be amplified. Amplification typically is performed on nucleic acids provided with adapters comprising primer hybridization sequences. Double-stranded nucleic acids can be amplified by any known form of amplification. This includes, without limitation, polymerase chain reaction (PCR) amplification, quantitative PCR, rolling circle amplification, multiple displacement amplification, loop-mediated isothermal amplification (LAMP), reverse transcription loop-mediated isothermal amplification (RT-LAMP), strand-displacement amplification (SDA), helicase-dependent amplification (HDA), or transcription-mediated amplification (TMA). For ease of description, reactions will be discussed in terms of PCR; necessary adjustments for other methods of amplification will be readily apparent to one of skill in the art.

### III. Nucleic Acid Analysis

Double-stranded nucleic acid molecules, whether amplified or not, may now be subject to analysis.

A. Nucleic Acid Sequencing

In one embodiment, double-stranded nucleic acids are analyzed by nucleic acid sequencing. Typically, nucleic acids are sequenced using high throughput sequencing. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing (PacBio), and nanopore DNA sequencing (e.g., Oxford Nanopore).

B. Analysis of Nucleic Acid Sequences

Nucleic acid sequencing produces sequence reads. Sequence reads are typically analyzed by mapping the sequence reads to a reference genome. For example, the current human genome reference sequence is hg38, which can be accessed at, for example, the NCBI website. A genetic locus for analysis can be a single nucleotide position in the genome, or a sequence or area of the genome, such as a gene, including surrounding areas such as promoter regions, or a chromosome.

After mapping sequences to a reference genome the results can be analyzed in a number of ways. One method of analysis is referred to as “peak analysis”. In this method the number of sequence reads mapping to loci across the reference genome can be determined. Because the nucleic acids have been enriched for sequences comprising modified nucleotides, loci to which many sequence reads appear as “peaks” of reads, for example, in a graph in which the X axis represents the genome and the “Y” axis represents the number of reads mapping thereto. Peaks can represent loci of nucleotide modification.

Another method involves single base resolution analysis. In this method, sequence reads are compared against a reference genome, using a single nucleotide as a locus. Cytosine form nucleotides that were converted to non-cytosine form nucleotides will appear as mismatches against the reference genome. For example, a cytosine residue in the reference genome would match with a thymidine residue in the sequence read. Cytosine residues in the reference genome that match with cytosine residues in the sequence reads represent target modified nucleotides.

C. DNA Microarray Analysis

In some embodiments, nucleic acids prepared by the methods described herein can be analyzed using a DNA microarray. DNA microarrays can be used for comparative genomic hybridization, chromatin immunoprecipitation analysis, and SNP detection. DNA micorarrays, also referred to as “DNA chips” are solid supports to which are attached positionally defined and addressable oligonucleotide probes. When sample nucleic acids are contacted with the array of nucleic acid probes, the sample nucleic acids hybridize to probes having complementary, or nearly complementary, sequences. The locations where sample nucleic acids have hybridized can be determined. This information can then be used to determine the identity or the sequence of the sample nucleic acids. Because they can detect nucleic acid molecules in a sequence-specific manner, DNA microarrays are useful for detecting sequences altered such that bases that read as “C” in a reference genome, are replaced by “T” after being treated by the methods described herein. DNA microarrays can be prepared in the lab, or purchased from, for example, Affymetrix (ThermoFisher).

D. Other Detection Methods

Other methods also can be used to detect nucleic acids. These methods can be done during an amplification process, and could be used as a readout for anchor-based bisulfite enrichment.

1. TaqMan

In TaqMan probe detection, a probe for a target DNA molecule comprises a fluorophore and a quencher moiety. During PCR, Taq polymerase that is extending a primer on the target DNA uses its 5′-3′ exonuclease activity to cleave a nucleotide from the hybridized TaqMan probe, thereby releasing the fluorophore. Once separated from the quencher, the fluorophore emits detectable florescent light.

2. Molecular Beacons

A molecular beacon is a nucleic acid in the form of a stem and loop structure. The stem is formed by complementary nucleotides at the termini of the molecule. Typically, a fluorophore is attached to the 5′ and of the molecule and a quencher is attached to the 3′ and of the molecule. The loop of the beacon comprises a nucleotide sequence complementary to a target nucleotide sequence in a target molecule. Upon hybridization of the Beacon with a molecule having the target sequence, the fluorophore and quencher are physically separated, producing a detectable fluorescence.

3. Padlock Probes and Molecular Inversion Probes

Padlock probes and molecular inversion probes are single-stranded nucleic acid molecules in which the termini comprise sequences that are complementary to a target molecule. In targeted bisulfite sequencing with padlock probes, padlock probes are provided. Each padlock probe has a common linker sequence flanked by two target-specific capturing arms. The linker sequence contains priming sites for universal primers. Multiple padlock probes cover a CpG island on partially overlapping regions on alternate DNA strands. A library of padlock probes is annealed to bisulfite-converted genomic DNA and the 3′ ends are extended and ligated with the 5′ and after removal of linear DNAs with exonuclease's, all circularized padlock probes are PCR-amplified using a pair of common primers. In a molecular inversion probe the termini bind to the target nucleic acid molecules leaving a gap, for example, a single base gap.

Molecular inversion probes can comprise termini having sequences complementary to target regions in the target nucleic acid, a pair of PCR primer binding sites, typically separated by a probe release cleavage site, a tag sequence for hybridization-based detection and a tag-release cleavage site. Upon hybridization to a target nucleic acid, the gap in the hybridization site can be filled by a ligase or a polymerase and ligase. Cleavage of the probe release site produces a single-stranded probe. PCR from the PCR primer sites in the probe amplify the target sequence and the capture sequence. Amplified molecules can be isolated by enrichment using the tag sequence. The tag sequence can be subsequently released.

4. qPCR

In another method, sequences are detected by qPCR. In qPCR, DNA is amplified by PCR in which detectably labeled nucleotides are incorporated into the amplified product. The rate and amount of label detected indicates the amount of target in the sample.

### IV. Diagnostic Methods

Anchored base enrichment of nucleic acid molecules treated to modify targeted/non-targeted bases can be used in diagnostic methods that involve detection of modified bases as biomarkers. In methods of discovering biomarkers, samples from two groups of subjects, one with a condition to be diagnosed, and the other without the condition, are provided. The condition can be any pathological condition including, without limitation, genetic conditions, cancers, age-related conditions such as progeria or accelerated aging, cellular pathologies, neuronal pathologies, etc.

Methods as described herein are used to produce genetic analysis of base modification patterns in each of the samples of each of the different groups. This genetic analysis can take the form of sequence information. The data is collected into a dataset and subject to statistical analysis to generate a model that distinguishes between the two groups. Any statistical method known in the art can be used for this purpose. Such methods, or tools, include, without limitation, correlational, Pearson correlation, Spearman correlation, chi-square, comparison of means/variances (e.g., paired T-test, independent T-test, ANOVA) regression analysis (e.g., simple regression, multiple regression, linear regression, non-linear regression, logistic regression, polynomial regression, stepwise regression, ridge regression, lasso regression, elastic net regression) or non-parametric analysis (e.g., Wilcoxon rank-sum test, Wilcoxon sign-rank test, sign test). Such tools are included in commercially available statistical packages such as MATLAB, JMP Statistical Software and SAS. Such methods produce models or classifiers which one can use to classify a particular biomarker profile into a particular state. Statistical analysis can be operator implemented or implemented by machine learning. The result of such analysis is a model that uses information about the location of modified bases, e.g., modified cytosine residues, to classify a subject from which a sample is taken as having or not having the condition.

Once a model for diagnosing a condition is established, the model can be used for diagnosis of a subject. In such methods, a sample comprising nucleic acids from the subject is provided. The nucleic acids are subject to the methods as described herein. Treated nucleic acids are analyzed to generate characteristic data, such as sequence data. The model is applied to the sequence data to classify the sample into the appropriate category.

For example, methods of detection can comprise (1) providing DNA from a biological sample from a subject; (2) generating double-stranded nucleic acid molecules enriched for sequences comprising modified cytosine residues using anchored base second strand synthesis as described herein; (3) mapping the location of modified cytosine residues in the double-stranded molecules that function as biomarkers to genetic loci. The presence of the biomarker is an indication of the condition to which the biomarker is associated.

The methods can involve any of the mapping strategies described herein. Furthermore, detection can be done by any method known in the art for detecting particular nucleotide sequences, including, but not limited to DNA sequencing, PCR, qPCR, hybridization of labeled probes against the biomarker, TaqMan amplification, or detection by molecular beacon.

### Exemplary Embodiments

Exemplary embodiments of the invention include, but are not limited to:

1. A method comprising:
a) chemically or enzymatically converting non-target forms of cytosine and/or modified cytosine in target nucleic acid molecules in a sample to non-cytosine residues to produce converted nucleic acid molecules;
b) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25; and
c) analyzing the double stranded nucleic acid molecules.

2. The method of embodiment 1, wherein the n=5 to 20, or 4 to 9, or 5.

3. The method of embodiment 1, wherein the primers are hexamers.

4. The method of embodiment 1, wherein X can be any of N, H, I, Q or J.

5. The method of embodiment 1, wherein XnG or X(n−1)CG are selected from NnG or N(n−1)CG, HnG or H(n−1)CG, InG or I(n−1)CG, QnG or Q(n−1)CG, JnG or J(n−1)CG or combinations thereof.

6. The method of embodiment 1, wherein XnG is 5′-NNNNNG-3′ or 5′-HHHHHG-3′, and X(n−1)CG is 5′-NNNNCG-3′ or 5′-HHHHCG-3′.

7. The method of embodiment 1, wherein the primers are hexamers.

8. The method of any of embodiments 1-7, wherein the set of primers is fully degenerate for the sequence XnG or X(n−1)CG.

9. The method of embodiment 1, wherein the target nucleic acid molecules comprise human DNA.

10. The method of embodiment 1, wherein the nucleic acids are from a pathological tissue or cell, e.g., a cancerous cells.

11. The method of embodiment 1, wherein the target nucleic acid molecules comprise purified DNA or RNA, or chromatin.

12. The method of embodiment 1, wherein the target nucleic acids have lengths between about 150 nucleotides and about 700 nucleotides.

13. The method of embodiment 1, wherein chemically or enzymatically converting comprises treatment with one or more of bisulfite, a Ten-Eleven-Translocation methylcytosine dioxygenase enzyme (“TET”) and an enzyme of the AID/APOBEC-class of enzymes (e.g., APOBEC3A (“A3A”)).

14. The method of embodiment 1, wherein target forms of cytosine comprise one or more of 5 methylcytosine (“5mC”), 5 hydroxymethylcytosine (“5hmC”), 5 formylcytosine (“5fC”) and 5 carboxylcytosine (“5caC”).

15. The method of embodiment 1, wherein chemically or enzymatically converting comprises converting cytosine forms other than 5mC and 5hmC to uracil.

16. The method of embodiment 1, wherein chemically or enzymatically converting comprises converting cytosine forms other than 5hmC to uracil.

17. The method of embodiment 1, wherein chemically or enzymatically converting comprises converting cytosine to uracil, but not converting 5mC, 5hmC, 5fC or 5caC to uracil.

18. The method of embodiment 1, wherein the non-cytosine residue is uracil.

19. The method of embodiment 1, wherein the primer comprises DNA, RNA, LNA, or PNA.

20. The method of embodiment 1, wherein the primer comprises a modified ribose or deoxyribose.

21. The method of embodiment 1, wherein the primer comprises a modified sugar residue that alters the melting temperature of the primer.

22. The method of embodiment 1, wherein the primer further comprises adapter and/or universal priming sequences.

23. The method of embodiment 22, wherein the adapter sequences comprise P3 and P5.

24. The method of embodiment 22, wherein the adapter sequences comprise P3 and P5.

25. The method of embodiment 1, wherein the primers comprise a sample barcode sequence.

26. The method of embodiment 1, wherein the primers comprise a molecular barcode sequence.

27. The method of embodiment 1, wherein the primer further comprises adapter and/or universal priming sequences.

28. The method of embodiment 1, wherein second strand synthesis is performed with a mesophilic or a thermophilic DNA polymerase.

29. The method of embodiment 1, wherein second strand synthesis is performed with an exo-polymerase.

30. The method of embodiment 1, wherein second strand synthesis is performed with a polymerase selected from Klenow exo-polymerase, Klenow polymerase, T4 DNA polymerase, Taq polymerase, pfu polymerase, DNA polymerase I, Phi29 polymerase and a reverse transcriptase (e.g., Moloney Murine Leukemia Virus (M-MLV), Avian Myeloblastosis Virus (AMV), and their mutated/altered versions.

31. The method of embodiment 1, wherein the primer is biotinylated in the method further comprises capturing double-stranded nucleic acid molecules comprising biotin.

32. The method of embodiment 31, further comprising introducing a 3′ terminal azide (N3) group to the nucleic acid molecule; attaching an alkylated adapter through a 5′-3-triazole bond to produce an adapter-tagged molecule; and amplifying the adapter-tagged molecule using a set of primers complementary to the 5′ and 3′ ends of the molecule.

33. The method of embodiment 1, comprising, after primer extension, attaching sequencer-specific adapters to the nucleic acid molecules to produce adapter-tagged nucleic acid molecules.

34. The method of embodiment 33, wherein attaching comprises end repair, optional addition of a nucleotide overhang, and blunt end or overhang ligation of the adapters.

35. The method of embodiment 33, wherein the adapters are specific for sequencing by Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing, and nanopore DNA sequencing.

36. The method of embodiment 1, wherein the double-stranded molecules are provided with primer hybridization sequences and the method comprises amplifying the double stranded nucleic acid molecules.

37. The method of embodiment 1, further comprising sequence capture of nucleic acids comprising target nucleotide sequences.

38. The method of embodiment 1, wherein analyzing comprises sequencing the double-stranded nucleic acid molecules, with or without nucleic acid amplification, to produce sequence reads.

39. The method of embodiment 38, wherein sequencing is performed by Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing, or nanopore DNA sequencing.

40. The method of embodiment 39, wherein analysis comprises peak analysis or SNP analyses.

41. The method of embodiment 39, comprising mapping the sequence reads to a reference genome.

42. The method of embodiment 41, further comprising mapping the genetic locus of one or more cytosine residues in the sequence reads that map to cytosine residues in reference genome and/or mapping the genetic locus of one or more thymidine residues in the sequence reads that map to cytosine residues in the reference genome, wherein a cytosine residue in a sequence read that maps to a cytosine residue in the reference genome represents a modified cytosine residue in the nucleic acid molecule sequenced to produce the sequence read.

43. The method of embodiment 1, wherein analyzing comprises DNA array analysis.

44. The method of embodiment 1, wherein the nucleic acid comprises RNA and second strand synthesis uses dUTP nucleotides.

45. The method of embodiment 1, wherein target DNA molecules are provided by:
i) providing a sample comprising chromatin (optionally in a cell);
ii) crosslinking proteins to DNA in the chromatin; optionally fragmenting the cross-linked chromatin; and
iii) isolating target nucleic acid molecules from the chromatin, by chromatin immunoprecipitation (ChIP).

46. The method of embodiment 45, wherein the immunoprecipitation targets nucleic acid sequences bound with a histone, a DNA polymerase, an RNA polymerase, methyl-binding proteins, or bound with a protein containing the following domains: bZIP domain, DNA-binding domain, helix-loop-helix, helix-turn-helix, MG-box, leucine zipper, lexitropsin, nucleic acid simulations, zinc finger, histone methylases, recruitment proteins, Swi6.

47. The method of embodiment 1, wherein target DNA molecules are provided by:
i) providing a sample comprising chromatin;
ii) crosslinking proteins to DNA in the chromatin (e.g., with formaldehyde);
iii) digesting chromatin to create fragmented chromatin;
iv) introducing biotin into the fragmented chromatin to produce biotinylated chromatin;
v) ligating the biotinylated chromatin fragments;
vi) decrosslinking, extracting and shearing the ligated fragments; and
vii) isolating the biotinylated sheared fragments.

48. A method of mapping non-bisulfite reactive cytosines in DNA comprising:
a) providing a sample comprising nucleic acid molecules, optionally fragmented;
b) treating the nucleic acid molecules with bisulfite, wherein treating converts unmodified cytosine residues to uracil;
c) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, X is any base, and n=2 to 25;
d) performing end repair and adapter ligation on the double-stranded nucleic acid molecules to produce adapter-tagged nucleic acid molecules;
e) amplifying (e.g., by PCR or qPCR), the adapter-tagged nucleic acid molecules; and
f) sequencing the amplified nucleic acid molecules.

49. The method of embodiment 48, wherein XnG is 5′-NNNNNG-3′ or 5′-HHHHHG-3′, and X(n−1)CG is 5′-NNNNCG-3′ or 5′-HHHHCG-3′.

50. A method comprising:
a) providing a sample comprising nucleic acid molecules, optionally fragmented;
b) protecting 5-hydroxymethylcytosine (“5hmC”) residues in the nucleic acid molecules;
c) converting 5-methylcytosine (“5mC”) and/or 5-formylcytosine (“5fC”) into 5-carboxylcytosine (“5caC”) residues;
d) converting C, and 5caC residues in the nucleic acids into uracil;
e) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, and X is any base, and n=2 to 25;
f) attaching adapters to the double-stranded nucleic acid molecules; to produce adapter-tagged nucleic acid molecules;
g) amplifying (e.g., by PCR, the adapter-tagged nucleic acid molecules; and
h) sequencing the amplified nucleic acid molecules.

51. The method of embodiment 50, wherein XnG is 5′-NNNNNG-3′ or 5′-HHHHHG-3′, and X(n−1)CG is 5′-NNNNCG-3′ or 5′-HHHHCG-3′.

52. The method of embodiment 50, wherein 5mC and/or 5fC are converted to 5caC by treatment with TET.

53. The method of embodiment 50, wherein 5hmC is protected by glucosylation, e.g., using T4 glucosyltransferase.

54. A method comprising:
a) providing a sample comprising nucleic acid molecules, optionally fragmented;
b) converting 5-methylcytosine (“5mC”), 5-hydroxymethylcytosine (“5hmC”) and/or 5-formylcytosine (“5fC”) into 5-carboxylcytosine (“5caC”) residues;
c) converting C residues in the nucleic acids into uracil, e.g., with an enzyme of the APOBEC/AID-class of enzymes;
d) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25;
e) attaching adapters to the double-stranded nucleic acid molecules; to produce adapter-tagged nucleic acid molecules;
f) amplifying (e.g., by PCR, the adapter-tagged nucleic acid molecules; and
g) analyzing the amplified nucleic acid molecules, e.g., by sequencing or by DNA array analysis.

55. The method of embodiment 54, wherein XnG is 5′-NNNNNG-3′ or 5′-HHHHHG-3′, and X(n−1)CG is 5′-NNNNCG-3′ or 5′-HHHHCG-3′.

56. A kit comprising:
(a) a set of primers comprising a nucleotide sequence wherein the primers comprise a nucleotide sequence 5′-XnG-3′ and/or 5′-X(n−1)CG-3′, wherein X is any base, and n=2 to 25;
(b) one or more containers, each container containing one of (i) sodium bisulfite, (2) Ten-Eleven Translocation methylcytosine dioxygenase 1 (“TET1”), T4 beta-glucosyl-transferase, APOBEC3A (“A3A”) or an enzyme from the AID/APOBEC class of deaminases.

57. The method of embodiment 56, wherein XnG is 5′-NNNNNG-3′ or 5′-HHHHHG-3′, and X(n−1)CG is 5′-NNNNCG-3′ or 5′-HHHHCG-3′.

58. The kit of embodiment 56, comprising TET1 from human, mouse, or invertebrate (e.g. Naegleria, Drosophila);

59. The kit of embodiment 56, wherein “X” includes at least one universal base, e.g., selected from (deoxy)inosine, nebularine, 3-Nitropyrrole, 5-Nitroindole.

60. A kit comprising:
(a) a set of primers comprising a nucleotide sequence 5′-XnG-3′ and/or 5′-X(n−1)CG-3′, wherein X is any base, and n=2 to 25;
(b) nucleic acid molecules in which at least one, but not all, forms of cytosine or modified cytosine in target nucleic acid molecules that are converted to uracil.

61. The method of embodiment 60, wherein XnG is 5′-NNNNNG-3′ or 5′-HHHHHG-3′, and X(n−1)CG is 5′-NNNNCG-3′ or 5′-HHHHCG-3′.

62. A kit comprising:
(a) a set of primers comprising a nucleotide sequence 5′-XnG-3′ and/or 5′-X(n−1)CG-3′, wherein X is any base, and n=2 to 25, wherein the primers comprises a tag, e.g., biotin;
(b) 3′-Azido-ddGTP;
(c) a 5′ alkyl oligo; and
(d) nucleic acid molecules in which at least one, but not all, forms of cytosine or modified cytosine in target nucleic acid molecules that are converted to

63. A composition comprising:
a) population single-stranded nucleic acid molecules; and
b) hybridized thereto, a set of primers comprising a nucleotide sequence 5′-HnG-3′ and/or 5′-H(n−1)CG-3′, wherein X is any base, and n=2 to 25.

64. The method of embodiment 63, wherein XnG is 5′-NNNNNG-3′ or 5′-HHHHHG-3′, and X(n−1)CG is 5′-NNNNCG-3′ or 5′-HHHHCG-3′.

65. A method of generating a model to classify a sample as pathological or nonpathological, comprising:
a) providing a first set of nucleic acid molecules from a first set of subjects having the pathology, and a second set of nucleic acid molecules from a second set of subjects not having the pathology;
b) treating nucleic acid molecules in the samples by:
(i) chemically or enzymatically converting non-target forms of cytosine and/or modified cytosine in target nucleic acid molecules in a sample to non-cytosine residues to produce converted nucleic acid molecules;
(ii) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25; and
c) analyzing the double stranded nucleic acid molecules to produce data mapping base modifications in the samples;
d) performing statistical analysis on the data to compare differences in base modification positions in the samples, wherein the statistical analysis produces a model that classifies a sample as pathological or nonpathological.

66. The method of embodiment 65, wherein XnG is 5′-NNNNNG-3′ or 5′-HHHHHG-3′, and X(n−1)CG is 5′-NNNNCG-3′ or 5′-HHHHCG-3′.

67. A method comprising:
(a) providing DNA from a biological sample from a subject;
(b) chemically or enzymatically converting non-target forms of cytosine and/or modified cytosine in target nucleic acid molecules in a sample to non-cytosine residues to produce converted nucleic acid molecules;
(c) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25;
(d) generating double-stranded nucleic acid molecules enriched for sequences comprising modified cytosine residues using an anchored base second strand synthesis method as described herein; and
(e) mapping the location of modified cytosine residues in the double-stranded molecules to genetic loci.

68. The method of embodiment 67, wherein XnG is 5′-NNNNNG-3′ or 5′-HHHHHG-3′, and X(n−1)CG is 5′-NNNNCG-3′ or 5′-HHHHCG-3′.

69. The method of embodiment 67, wherein the mapped modified cytosine residue is a biomarker.

### Examples

### I. AB-BS (Also Referred as ABBS or ABBA)

This method takes advantage of the fact that 5mC and 5hmC bases present in DNA or RNA do not react with bisulfite whereas unmodified cytosines, 5-formylcytosine and 5-carboxycytosine (and potentially other, still to be identified, modified cytosines), are deaminated and efficiently converted to uracil. These uracil sites, upon synthesis of a second strand with Klenow exo-polymerase, base-pair with adenine; thus, any bisulfite-reactive Cs in the original parent strain of DNA are converted to uracil and read out as Ts in PCR and/or sequencing. Using this, our invention enables amplification of DNA from any unreacted cytosine present in the genome (e.g., 5mC and 5hmC) using a random priming strategy during second strand synthesis in which primers have the following structure 5′-HHHHHG-3′ (where H=not G) (or 5′-HHHHCG-3′, to enrich for CpG methylation specifically), or 5′-NNNNNG-3′ (where N=A, C, G, T/U) (or 5′-NNNNCG-3′, to enrich for CpG methylation specifically). The terminal 3′ G will anchor the primer at any C that did not react with bisulfite and the internal and 5′ H, if any, will avoid that the primer partially hybridizes to C. Thus PCR amplification driven from these anchored primers will preferentially amplify regions of the genome that are methylated and/or hydroxymethylated.

### Protocol

1/500 ng of DNA spiked with 0.5% of unmethylated lambda DNA (to measure efficient bisulfite conversion), bisulfite convert with EZ DNA methylation lightning kit (Zymo Research Corp.) following the protocol.

2/Nanodrop quantification.

3/Second strand synthesis:

| | | |
|---|---|---|
| 10× NEBuffer2 | 5 | μL |
| 10 mM dNTPs | 1.25 | μL |
| Water | 3.75 | μL |
| 50 ng/μL 5′-HHHHHG-3′oligo | 25 | μL |
| 75 ng Bisulfite-treated sample DNA | 10 | μL |

1. 94° C. for 5 min
2. 4° C. for 5 min 4 After 5′ add 5 μL of Klenow exo-(NEB, 5U/μL) to the solution and mix well.
3. Gradual increment from 4° C. to 37° C. at a rate of 5%.
4. 37° C. for 60 min
5. 75° C. for 15 min, then 4° C./∞

4/Purify dsDNA using a MinElute column from Qiagen, 2 washings, eluting in 20 μL of tris-HCl pH 8.0 10 mM, then quantification with Qubit 2.0 dsDNA HS kit.

5/Library 2s Swift with 2.5 ng starting material.

### Additional Examples

### II. AB-TAB-Seq (Anchored Base Tet-Assisted Bisulfite Sequencing).

Steps:
1. 5hmC bases are first protected via glucosylation with recombinant T4 phage beta-glucosyltransferase (Active Motif cat #81249) using conditions outlined in Active Motif's Hydroxymethyl Collector-Seq kit (#55019).
2. 5mC, and 5fC are oxidized by recombinant TET1 enzyme (Active Motif's cat #81148) to form 5-carboxylcytosine while the glucosylated 5hmC remains intact.
3. Oxidized DNA is then reacted with bisulfite to deaminate unmodified cytosines and 5-carboxycytosine groups throughout the genome.
4. DNA is then purified (Active Motif's ChIP IP DNA Purification Kit) and processed as above with Klenow exo-, anchored oligos, and double-stranded DNA library preparation.
5. NGS of formed libraries to identify enriched regions of 5hmC from the genome.

### III. AB-A3A-Seq (Anchored Base A3A-Assisted Sequencing). See Drawings

Steps:
1. DNA is treated with recombinant TET1 enzyme to convert 5mC, 5hmC, 5fC bases to 5caC while unmodified cytosines remain intact.
2. TET1-oxidized DNA is then treated with recombinant APOBEC3A (A3A) to deaminate unmodified cytosines, converting these bases to uracil. All TET1-formed 5caC sites remain unaffected by A3A.
3. DNA is then purified (Active Motif's ChIP IP DNA Purification Kit) and processed as above with Klenow exo-, anchored oligos, and double-stranded DNA library preparation.
4. NGS of formed libraries to identify enriched regions of modified, TET-oxidized bases from the genome.

### IV. AB-HiC

In this embodiment, DNA that was used in “HiC” (to map interacting loci), e.g. Lieberman-Aiden et al., Science (2009) Vol. 326, Issue 5950, pp. 289-293, is subjected to fragmentation and heat-denaturation. Then, a mesophilic polymerase synthetizes a second strand using short primers containing a motif consensus (anchored at a motif consensus). (In this proposal, NNNNNG or HHHHHG are emphasized, but one could use any primer as described herein, and that can make double-stranded DNA used for library prep, as exemplified here with a motif.) After sequencing and filtering the reads that are outside of targeted genomic locations (in a Brower Extensible Data “BED” file http://genome.ucsc.edu/FAQ/FAQformat#format1), specific interactions are called. This method is significantly cheaper compared to regular HiC (for which ˜1 billion reads are usually needed). In that specific case, a primer containing a hexamer for example would reduce the sequencing costs by several hundred folds.

The isolated nucleic acids are analyzed. Analysis could involve, for example, nucleic acid sequencing, PCR, qPCR and the like. Generally sequenced for subsequent analysis. The methods described herein generally employ high throughput sequencing methods. As used herein, the term “high throughput sequencing” refers to the simultaneous or near simultaneous sequencing of thousands of nucleic acid molecules. High throughput sequencing is sometimes referred to as “next generation sequencing” or “massively parallel sequencing.” Platforms for high throughput sequencing include, without limitation, massively parallel signature sequencing (MPSS), Polony sequencing, 454 pyrosequencing, Illumina (Solexa) sequencing, SOLiD sequencing, Ion Torrent semiconductor sequencing, DNA nanoball sequencing (Complete Genomics), Heliscope single molecule sequencing, single molecule real time (SMRT) sequencing (PacBio), and nanopore DNA sequencing (e.g., Oxford Nanopore). Nucleotide sequences of nucleic acids produced by sequencing are referred to herein as “sequence information”, “sequence reads” or “sequence data”.

HiC: We briefly summarize the process: cells are crosslinked with formaldehyde; DNA is digested with a restriction enzyme that leaves a 5′ overhang; the 5′ overhang is filled, including a biotinylated residue; and the resulting blunt-end fragments are ligated under dilute conditions that favor ligation events between the cross-linked DNA fragments (in situ ligation in permeabilized cells is also an option). The resulting DNA sample contains ligation products consisting of fragments that were originally in close spatial proximity in the nucleus, marked with biotin at the junction. A HiC library is created by shearing the DNA and selecting the biotin-containing fragments with streptavidin beads. The library is then analyzed by using massively parallel DNA sequencing, producing a catalog of interacting fragments.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
a) chemically or enzymatically converting non-target forms of cytosine and/or modified cytosine in target nucleic acid molecules in a sample to non-cytosine residues to produce converted nucleic acid molecules;
b) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25; and
c) analyzing the double stranded nucleic acid molecules.

2. The method of claim 1, wherein the n=5 to 20, or 4 to 9, or 5.

3. The method of claim 1, wherein the primers are hexamers.

4. The method of claim 1, wherein X can be any of N, H, I, Q or J.

5. The method of claim 1, wherein XnG or X(n−1)CG are selected from NnG or N(n−1)CG, HnG or H(n−1)CG, InG or I(n−1)CG, QnG or Q(n−1)CG, JnG or J(n−1)CG or combinations thereof.

6. The method of claim 1, wherein XnG is 5′-NNNNNG-3′ or 5′-HHHHHG-3′, and X(n−1)CG is 5′-NNNNCG-3′ or 5′-HHHHCG-3′.

7. The method of claim 1, wherein the primers are hexamers.

8. The method of any of claims 1-7, wherein the set of primers is fully degenerate for the sequence XnG or X(n−1)CG.

9. The method of claim 1, wherein the target nucleic acid molecules comprise human DNA.

10. The method of claim 1, wherein the nucleic acids are from a pathological tissue or cell, e.g., a cancerous cells.

11. The method of claim 1, wherein the target nucleic acid molecules comprise purified DNA or RNA, or chromatin.

12. The method of claim 1, wherein the target nucleic acids have lengths between about 150 nucleotides and about 700 nucleotides.

13. The method of claim 1, wherein chemically or enzymatically converting comprises treatment with one or more of bisulfite, a Ten-Eleven-Translocation methylcytosine dioxygenase enzyme (“TET”) and an enzyme of the AlD/APOBEC-class of enzymes (e.g., APOBEC3A (“A3A”)).

14. The method of claim 1, wherein target forms of cytosine comprise one or more of 5 methylcytosine (“5mC”), 5 hydroxymethylcytosine (“5hmC”), 5 formylcytosine (“5fC”) and 5 carboxylcytosine (“5caC”).

15. The method of claim 1, wherein chemically or enzymatically converting comprises converting cytosine forms other than 5mC and 5hmC to uracil.

16. The method of claim 1, wherein chemically or enzymatically converting comprises converting cytosine forms other than 5hmC to uracil.

17. The method of claim 1, wherein chemically or enzymatically converting comprises converting cytosine to uracil, but not converting 5mC, 5hmC, 5fC or 5caC to uracil.

18. The method of claim 1, wherein the non-cytosine residue is uracil.

19. The method of claim 1, wherein the primer comprises DNA, RNA, LNA, or PNA.

20. The method of claim 1, wherein the primer comprises a modified ribose or deoxyribose.

21. The method of claim 1, wherein the primer comprises a modified sugar residue that alters the melting temperature of the primer.

22. The method of claim 1, wherein the primer further comprises adapter and/or universal priming sequences.

23. The method of claim 22, wherein the adapter sequences comprise P3 and P5.

24. The method of claim 22, wherein the adapter sequences comprise P3 and P5.

25. The method of claim 1, wherein the primers comprise a sample barcode sequence.

26. The method of claim 1, wherein the primers comprise a molecular barcode sequence.

27. The method of claim 1, wherein the primer further comprises adapter and/or universal priming sequences.

28. The method of claim 1, wherein second strand synthesis is performed with a mesophilic or a thermophilic DNA polymerase.

29. The method of claim 1, wherein second strand synthesis is performed with an exo-polymerase.

30. The method of claim 1, wherein second strand synthesis is performed with a polymerase selected from Klenow exo-polymerase, Klenow polymerase, T4 DNA polymerase, Taq polymerase, pfu polymerase, DNA polymerase I, Phi29 polymerase and a reverse transcriptase (e.g., Moloney Murine Leukemia Virus (M-MLV), Avian Myeloblastosis Virus (AMV), and their mutated/altered versions.

31. The method of claim 1, wherein the primer is biotinylated in the method further comprises capturing double-stranded nucleic acid molecules comprising biotin.

32. The method of claim 31, further comprising introducing a 3′ terminal azide (N3) group to the nucleic acid molecule; attaching an alkylated adapter through a 5′-3-triazole bond to produce an adapter-tagged molecule; and amplifying the adapter-tagged molecule using a set of primers complementary to the 5′ and 3′ ends of the molecule.

33. The method of claim 1, comprising, after primer extension, attaching sequencer-specific adapters to the nucleic acid molecules to produce adapter-tagged nucleic acid molecules.

34. The method of claim 33, wherein attaching comprises end repair, optional addition of a nucleotide overhang, and blunt end or overhang ligation of the adapters.

35. The method of claim 33, wherein the adapters are specific for sequencing by Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing, and nanopore DNA sequencing.

36. The method of claim 1, wherein the double-stranded molecules are provided with primer hybridization sequences and the method comprises amplifying the double stranded nucleic acid molecules.

37. The method of claim 1, further comprising sequence capture of nucleic acids comprising target nucleotide sequences.

38. The method of claim 1, wherein analyzing comprises sequencing the double-stranded nucleic acid molecules, with or without nucleic acid amplification, to produce sequence reads.

39. The method of claim 38, wherein sequencing is performed by Polony sequencing, 454 pyrosequencing, Illumina (Solexa) sequencing, SOLiD sequencing, Ion Torrent semiconductor sequencing, DNA nanoball sequencing, Heliscope single molecule sequencing, single molecule real time (SMRT) sequencing, or nanopore DNA sequencing.

40. The method of claim 39, wherein analysis comprises peak analysis or SNP analyses.

41. The method of claim 39, comprising mapping the sequence reads to a reference genome.

42. The method of claim 41, further comprising mapping the genetic locus of one or more cytosine residues in the sequence reads that map to cytosine residues in reference genome and/or mapping the genetic locus of one or more thymidine residues in the sequence reads that map to cytosine residues in the reference genome, wherein a cytosine residue in a sequence read that maps to a cytosine residue in the reference genome represents a modified cytosine residue in the nucleic acid molecule sequenced to produce the sequence read.

43. The method of claim 1, wherein analyzing comprises DNA array analysis.

44. The method of claim 1, wherein the nucleic acid comprises RNA and second strand synthesis uses dUTP nucleotides.

45. The method of claim 1, wherein target DNA molecules are provided by:
i) providing a sample comprising chromatin (optionally in a cell);
ii) crosslinking proteins to DNA in the chromatin; optionally fragmenting the cross-linked chromatin; and
iii) isolating target nucleic acid molecules from the chromatin, by chromatin immunoprecipitation (ChIP).

46. The method of claim 45, wherein the immunoprecipitation targets nucleic acid sequences bound with a histone, a DNA polymerase, an RNA polymerase, methyl-binding proteins, or bound with a protein containing the following domains: bZIP domain, DNA-binding domain, helix-loop-helix, helix-turn-helix, MG-box, leucine zipper, lexitropsin, nucleic acid simulations, zinc finger, histone methylases, recruitment proteins, Swi6.

47. The method of claim 1, wherein target DNA molecules are provided by:
i) providing a sample comprising chromatin;
ii) crosslinking proteins to DNA in the chromatin (e.g., with formaldehyde);
iii) digesting chromatin to create fragmented chromatin;
iv) introducing biotin into the fragmented chromatin to produce biotinylated chromatin;
v) ligating the biotinylated chromatin fragments;
vi) decrosslinking, extracting and shearing the ligated fragments; and
vii) isolating the biotinylated sheared fragments.

48. A method of mapping non-bisulfite reactive cytosines in DNA comprising:
a) providing a sample comprising nucleic acid molecules, optionally fragmented;
b) treating the nucleic acid molecules with bisulfite, wherein treating converts unmodified cytosine residues to uracil;
c) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, X is any base, and n=2 to 25;
d) performing end repair and adapter ligation on the double-stranded nucleic acid molecules to produce adapter-tagged nucleic acid molecules;
e) amplifying (e.g., by PCR or qPCR), the adapter-tagged nucleic acid molecules; and
f) sequencing the amplified nucleic acid molecules.

49. The method of claim 48, wherein XnG is 5′-NNNNNG-3′ or 5′-HHHHHG-3′, and X(n−1)CG is 5′-NNNNCG-3′ or 5′-HHHHCG-3′.

50. A method comprising:
a) providing a sample comprising nucleic acid molecules, optionally fragmented;
b) protecting 5-hydroxymethylcytosine (“5hmC”) residues in the nucleic acid molecules;
c) converting 5-methylcytosine (“5mC”) and/or 5-formylcytosine (“5fC”) into 5-carboxylcytosine (“5caC”) residues;
d) converting C, and 5caC residues in the nucleic acids into uracil;
e) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, and X is any base, and n=2 to 25;
f) attaching adapters to the double-stranded nucleic acid molecules; to produce adapter-tagged nucleic acid molecules;
g) amplifying (e.g., by PCR, the adapter-tagged nucleic acid molecules; and
h) sequencing the amplified nucleic acid molecules.

51. The method of claim 50, wherein XnG is 5′-NNNNNG-3′ or 5′-HHHHHG-3′, and X(n−1)CG is 5′-NNNNCG-3′ or 5′-HHHHCG-3′.

52. The method of claim 50, wherein 5mC and/or 5fC are converted to 5caC by treatment with TET.

53. The method of claim 50, wherein 5hmC is protected by glucosylation, e.g., using T4 glucosyltransferase.

54. A method comprising:
a) providing a sample comprising nucleic acid molecules, optionally fragmented;
b) converting 5-methylcytosine (“5mC”), 5-hydroxymethylcytosine (“5hmC”) and/or 5-formylcytosine (“5fC”) into 5-carboxylcytosine (“5caC”) residues;
c) converting C residues in the nucleic acids into uracil, e.g., with an enzyme of the APOBEC/AID-class of enzymes;
d) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25;
e) attaching adapters to the double-stranded nucleic acid molecules; to produce adapter-tagged nucleic acid molecules;
f) amplifying (e.g., by PCR, the adapter-tagged nucleic acid molecules; and
g) analyzing the amplified nucleic acid molecules, e.g., by sequencing or by DNA array analysis.

55. The method of claim 54, wherein XnG is 5′-NNNNNG-3′ or 5′-HHHHHG-3′, and X(n−1)CG is 5′-NNNNCG-3′ or 5′-HHHHCG-3′.

56. A kit comprising:
(a) a set of primers comprising a nucleotide sequence wherein the primers comprise a nucleotide sequence 5′-XnG-3′ and/or 5′-X(n−1)CG-3′, wherein X is any base, and n=2 to 25;
(b) one or more containers, each container containing one of (i) sodium bisulfite, (2) Ten-Eleven Translocation methylcytosine dioxygenase 1 (“TET1”), T4 beta-glucosyl-transferase, APOBEC3A (“A3A”) or an enzyme from the AID/APOBEC class of deaminases.

57. The method of claim 56, wherein XnG is 5′-NNNNNG-3′ or 5′-HHHHHG-3′, and X(n−1)CG is 5′-NNNNCG-3′ or 5′-HHHHCG-3′.

58. The kit of claim 56, comprising TET1 from human, mouse, or invertebrate (e.g. Naegleria, Drosophila).

59. The kit of claim 56, wherein “X” includes at least one universal base, e.g., selected from (deoxy)inosine, nebularine, 3-Nitropyrrole, 5-Nitroindole.

60. A kit comprising:
(a) a set of primers comprising a nucleotide sequence 5′-XnG-3′ and/or 5′-X(n−1)CG-3′, wherein X is any base, and n=2 to 25;
(b) nucleic acid molecules in which at least one, but not all, forms of cytosine or modified cytosine in target nucleic acid molecules that are converted to uracil.

61. The method of claim 60, wherein XnG is 5′-NNNNNG-3′ or 5′-HHHHHG-3′, and X(n−1)CG is 5′-NNNNCG-3′ or 5′-HHHHCG-3′.

62. A kit comprising:
(a) a set of primers comprising a nucleotide sequence 5′-XnG-3′ and/or 5′-X(n−1)CG-3′, wherein X is any base, and n=2 to 25, wherein the primers comprises a tag, e.g., biotin;
(b) 3′-Azido-ddGTP;
(c) a 5′ alkyl oligo; and
(d) nucleic acid molecules in which at least one, but not all, forms of cytosine or modified cytosine in target nucleic acid molecules that are converted to

63. A composition comprising:
a) population single-stranded nucleic acid molecules; and
b) hybridized thereto, a set of primers comprising a nucleotide sequence 5′-HnG-3′ and/or 5′-H(n−1)CG-3′, wherein X is any base, and n=2 to 25.

64. The method of claim 63, wherein XnG is 5′-NNNNNG-3′ or 5′-HHHHHG-3′, and X(n−1)CG is 5′-NNNNCG-3′ or 5′-HHHHCG-3′.

65. A method of generating a model to classify a sample as pathological or nonpathological, comprising:
a) providing a first set of nucleic acid molecules from a first set of subjects having the pathology, and a second set of nucleic acid molecules from a second set of subjects not having the pathology;
b) treating nucleic acid molecules in the samples by:
(i) chemically or enzymatically converting non-target forms of cytosine and/or modified cytosine in target nucleic acid molecules in a sample to non-cytosine residues to produce converted nucleic acid molecules;
(ii) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25; and
c) analyzing the double stranded nucleic acid molecules to produce data mapping base modifications in the samples;
d) performing statistical analysis on the data to compare differences in base modification positions in the samples, wherein the statistical analysis produces a model that classifies a sample as pathological or nonpathological.

66. The method of claim 65, wherein XnG is 5′-NNNNNG-3′ or 5′-HHHHHG-3′, and X(n−1)CG is 5′-NNNNCG-3′ or 5′-HHHHCG-3′.

67. A method comprising:
(a) providing DNA from a biological sample from a subject;
(b) chemically or enzymatically converting non-target forms of cytosine and/or modified cytosine in target nucleic acid molecules in a sample to non-cytosine residues to produce converted nucleic acid molecules;
(c) performing second strand synthesis on denatured, converted nucleic acid molecules by hybridizing a set of primers to the denatured, converted nucleic acid molecules and extending the primers to produce double stranded nucleic acid molecules;
wherein the primers comprise a nucleotide sequence 5′-XnG-3′ and/or 5′-X(n−1)CG-3′, wherein X is any base, and n=2 to 25;
(d) generating double-stranded nucleic acid molecules enriched for sequences comprising modified cytosine residues using an anchored base second strand synthesis method as described herein; and
(e) mapping the location of modified cytosine residues in the double-stranded molecules to genetic loci.

68. The method of claim 67, wherein XnG is 5′-NNNNNG-3′ or 5′-HHHHHG-3′, and X(n−1)CG is 5′-NNNNCG-3′ or 5′-HHHHCG-3′.

69. The method of claim 67, wherein the mapped modified cytosine residue is a biomarker.
